(19) United States
(12) Patent Application Publication
(10) Pub. No.: US 2009/0022821 A1
(43) Pub. Date: Jan. 22, 2009

(54) COMPOSITIONS FROM WAX MYRTLE FOR THE TREATMENT OF CANCER, CARDIOVASCULAR AND INFLAMMATORY DISEASES

(76) Inventors: Inderjit Kumar Dev, Durham, NC (US); Ven Subbiah, Garner, NC (US)

Correspondence Address: PASSE' INTELLECTUAL PROPERTY, LLC, 1717 BRASSFIELD RD., RALEIGH, NC, 27614, US

(21) Appl. No.: 12/174,865

(22) Filed: Jul. 17, 2008

Related U.S. Application Data
(60) Provisional application No. 60/950,277, filed on Jul. 17, 2007.

(51) Int. Cl. A61K 36/185; A61K 31/235

(52) U.S. Cl. 424/725; 514/533

(57) ABSTRACT

The present invention relates to the use of certain novel compositions from wax myrtle (Morella cerifera) which are inhibitors of nuclear factor kappa B (NF-kB), oxidative stress (activation of Nerf2) and the activity of the endothelin receptor. In particular, it relates to useful enriched fractions and pharmaceutical compositions from wax myrtle for use in the treatment of cardiovascular and inflammatory diseases and for cancers susceptible to an inhibitor of oxidative stress (activation of Nerf2), NF-kB inhibitor and an endothelin receptor inhibitor. The present invention also relates to compositions from wax myrtle useful to inhibit cell proliferation and for the induction of apoptosis.

This application claims priority of the U.S. Application Ser. No. 60/950,277 filed Jul. 17, 2007 and incorporated herein in its entirety by reference.

### FIELD OF THE INVENTION

This invention relates to the use of certain novel compositions from wax myrtle (Morella cerifera; synonym, Myrica cerifera) which are inhibitors of nuclear factor kappa B (NF-kB), oxidative stress (activation of Nerf2) and the activity of the endothelin receptor. In particular, it relates to useful enriched fractions and pharmaceutical compositions from wax myrtle for use in the treatment of cardiovascular and inflammatory diseases and for cancers susceptible to an inhibitor of oxidative stress (activation of Nerf2), NF-kB inhibitor and an endothelin receptor inhibitor. The present invention also relates to compositions from wax myrtle useful to inhibit cell proliferation and for the induction of apoptosis.

### BACKGROUND OF THE INVENTION

The U.S. Food and Drug Administration (FDA) has recently established a Botanical Drug Team as well as a set of guidelines for the development of complex botanical extracts as ethical pharmaceuticals with disease-related claims. Such changes in the FDA regulatory environment now allow for the development of botanicals at a much more rapid pace than new chemical entities or biologics, resulting in approval costs that may be reduced by an order of magnitude. Additionally, this new guidance provides for unique guarantees of market exclusivity for NDA botanicals as well as the acceptance of synergistic combinations of bioactives.

The synergistic components found in botanical mixtures represent a largely untapped source of new pharmaceutical products with novel and multiple mechanisms of action. Recent developments in plant biotechnology have created the tools to produce botanical mixtures at a level comparable to that of pure drug compounds, thus meeting the requirements of the FDA. Botanical drug products will ultimately compete alongside conventional pharmaceuticals in the $300 billion global pharmaceutical marketplace.

The present invention relates to methods for the preparation of standardized extracts from the leaves and twigs of wax myrtle that are enriched in many synergistic components. These compounds may inhibit oxidative stress, activation of NF-kB or endothelin receptor. The presence of the antagonistic activity towards oxidative stress, activation of NF-kB, and endothelin receptor in the same extract is synergistic and can lead to useful compositions suitable for the treatment and prevention of cancer, inflammation related diseases and cardiovascular diseases.

The wax myrtle is a member of the bayberry family, Myricaceae. Its botanical name was Myrica cerifera, however, it has recently been reclassified as Morella cerifera. The narrow, evergreen leaves are a glossy olive-green and are 2 to 4 inches long. They are quite aromatic when crushed, releasing a pleasant, spicy scent. The wax myrtle's native range includes the coastal plain and piedmont regions of the Southeast United States, from Virginia to Florida and west to Texas.

Native Americans took a decoction of the stems and leaves for fever. It was also used as astringent, circulatory stimulant, mild diaphoretic, and tonic. It was a key astringent used by the Physiomedical herbalists and was a major component of Thomson's composition powder. The wax from the berries was used in candle making, hence some of its common names. Used in Puerto Rico to treat stubborn ulcers. Root bark used historically in the United States as an astringent and in larger doses an emetic, for chronic gastritis, diarrhea, dysentery, leucorrhea, jaundice, fevers; externally for hard-to-heal ulcers.

In the current invention, the inventors have found that the extracts prepared from wax myrtle leaves and twigs have potent activity against validated molecular targets for inflammatory diseases, respiratory and cardiovascular diseases and cancer. These molecular targets include nuclear erythroid 2 p45-related factor 2 (Nerf2), nuclear factor kappa B (NF-kB) and endothelin receptor A (ETA). The endothelin receptors exist in various tissue and organs such as vessels, trachea and the like and their excessive stimulation can lead to circulatory diseases such as pulmonary arterial hypertension, acute and chronic heart failure, acute and chronic renal failure, atherosclerosis, cerebrovascular diseases and the like.

The family of NF-kB transcription factors comprises important regulatory proteins that impact virtually every feature of cellular adaptation, including responses to stress, inflammatory reactions, activation of immune cell function, cellular proliferation, programmed cell death (apoptosis), differentiation and oncogenesis. NF-kB regulates more than 150 genes, including cytokines, chemokines, cell adhesion molecules, and growth factors. It is therefore not surprising that diseases result when NF-kB -dependent transcription is not appropriately-regulated. NF-kB has been implicated in several pathologies, including certain cancers (e.g., Hodgkin's disease, breast cancer, and prostate cancer), diseases associated with inflammation (e.g., rheumatoid arthritis, asthma, inflammatory bowel disease (e.g., Crohn's disease and ulcerative colitis), alcoholic liver disease, non-alcoholic steatohepatitis, pancreatitis, primary dysmenorrhea, psoriasis, and atherosclerosis) and Alzheimer's disease. Inhibition of NF-kB also protects against chemotherapy-induced adverse effects including oral and GI mucositis, alopecia and neutropenia.

Oxidative stress contributes to the general decline in cellular functions that are associated with many human diseases including asthma, emphysema, Alzheimer disease, Parkinson disease, atherosclerosis, macular degeneration, degenerative retinal damage, rheumatoid arthritis, multiple sclerosis, muscular dystrophy, human cancers as well as the aging. To counteract oxidative stress, higher animals have developed elaborate mechanism, including phase II detoxifying enzymes and antioxidant proteins. Induction of phase 2 enzymes is predominantly mediated by a redox-sensitive transcription factor NF-E2 related factor-2 (Nrf2). A variety of phytochemicals are able to activate Nrf2 thereby upregulating a set of enzymes including NADP(H): quinone oxidoreductase-1 (NQO1), superoxide dismutase (SOD), glutathione S-transferase (GST), hemeoxygenase-1 (HO-1), and glutamyl cysteine ligase (GCL). Nrf2 is sequestered in the cytoplasm as an inactive complex with its cytosolic repressor Kelch-like ECH associated protein 1 (Keap1). Dissociation of Nrf2 from the inhibitory protein Keap1 is a prerequisite for nuclear translocation and subsequent DNA binding of Nrf2. The genetic ablation of the Nrf2 results in severe airway inflammation and development of emphysema and asthma in mice. The importance of Nrf2 activation for chemoprevention was evident from a remarkably higher incidence of benzo[α]pyrene-induced gastric neoplasia in Nrf2-deficient mice, which were less responsive to the phase 11 enzyme inducer oltipraz than did wild-type mice. Thus, Nrf2 plays a central role in the regulation of constitutive and inducible expression of phase 2 enzymes in vivo.

Oxidative stress may also have a fundamental role in enhancing inflammation through the upregulation of redox-sensitive transcription factors, such as NF-kB and activating protein 1 (AP-1). Studies in macrophage cell lines and alveolar and bronchial epithelial cells show that oxidants cause the release of inflammatory mediators, such as IL-8, IL-1, and NO, and that these events are associated with increased expression of the genes for these inflammatory mediators and increased nuclear binding or activation of NF-kB. The linking of NF-kB to its consensus site in the nucleus leads to enhanced transcription of proinflammatory genes and therefore inflammation, which itself will produce more oxidative stress, creating a vicious circle of enhanced inflammation resulting from the increased oxidative stress. Therefore, it is believed that inhibitors of NF-kB and inhibitors of oxidative stress (activation of Nerf2) would work in concert and produce synergistic effects.

It is also believed that the presence of endothelin receptor and NF-kB antagonistic activity on the same molecule can be synergistic due to several reasons. First, reductions in endothelin levels due to the inhibition of gene transcription by NFkB will make inhibition of endothelin receptor more effective. Most endothelin receptor antagonists compete with endothelin for receptor binding; thus inhibition of endothelin receptor antagonists in the presence of reduced concentrations of endothelin should be enhanced substantially. Second, the effects of endothelin receptor antagonists and NF-kB antagonists on the apoptotic pathways complement each other. The inhibition of NF-kB induces apoptosis by regulating gene transcription of anti-apoptotic genes; whereas, endothelin acts as an antiapoptotic factor, modulating cell survival pathways through Bcl-2 and phosphatidylinositol 3-kinase/Akt pathways.

It would be highly desirable to enrich the fractions from wax myrtle that have activity against NF-kB, Nerf2 and endothelin receptor three medically important molecular targets. In particular, it would be desirable to provide the standardized leaves and twig extract from wax myrtle for use in a pharmaceutical, nutraceutical and cosmeceuticals compositions suitable for the treatment and prevention of cancer, inflammation related diseases, cardiovascular diseases, including coronary heart disease, fungal and bacterial infections, as an anticoagulant, enhancement of adenylate cyclase activity, anti-inflammatory, vasodilator, antimyocardiac, retardation of cholesterol biosynthesis, inhibition of lipoprotein oxidation, treatment of acne and eczema, prevent hair loss, whitening skin and protect myocardium from hypoxia-induced cardiac contractile failure.

### BRIEF DESCRIPTION OF THE DRAWINGS

FIGS. 1a and 1b is Finger Print of ENRICHED EXTRACT, LC-MS HPLC Profile: Intensity (mV, y-axis) retention times (x-axis).

FIG. 2 is a graph of a dose-dependent decrease in transactivation of NF-kB by ENRICHED EXTRACT

FIG. 3 is a graph of Growth Inhibition of Aortic Smooth Muscle Cells (A7r5) by ENRICHED EXTRACTWM extract.

FIG. 4 is a graph of an Induction of Histone-DNA Fragments in Aortic Smooth Muscle Cells (A7r5) by ENRICHED EXTRACT, SP 2011, Grown in Serum Free Medium.

### SUMMARY OF THE INVENTION

This invention relates to the use of certain novel compositions from wax myrtle (Morella cerifera), which are inhibitors of activation of NF-kB, oxidative stress (activation of Nerf2) and inhibit the activity of the endothelin receptor.

In particular, it relates to useful enriched fractions and pharmaceutical compositions from wax myrtle for use in the treatment of cardiovascular and inflammatory diseases and for cancers susceptible to an inhibitor of oxidative stress (activation of Nerf2), NF-kB inhibitor and an endothelin receptor inhibitor.

The present invention also relates to methods for preparing standardized extracts and botanical drugs from wax myrtle. The method includes assay guided steps that lead to the enrichment of several synergistic components from the leaves and twigs of wax myrtle with activity against multiple medically useful molecular targets. These compounds inhibit oxidative stress (activation of Nerf2), activation of NF-kB or endothelin receptor. The simultaneous inhibition of activation of NF-kB, oxidative stress and endothelin receptor is synergistic and can lead to useful compositions suitable for the treatment and prevention of cancer, inflammation related diseases and cardiovascular diseases.

The resulting isolated extract can be used in combination with a pharmaceutically acceptable carrier to provide a pharmaceutical composition suitable for use in treatment of a myriad of diseases. In one embodiment, the isolated extract can be used in the treatment of coronary heart disease.

### DETAILED DESCRIPTION OF THE INVENTION

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

The invention relates to methods for preparing standardized extracts from wax myrtle that use assay-guided fractionation of the plant leading to the enrichment of compounds that inhibit oxidative stress, activation of NF-kB or endothelin receptor. The simultaneous inhibition of activation of NF-kB, oxidative stress and endothelin receptor is synergistic and can lead to useful compositions suitable for the treatment and prevention of cancer, inflammation related diseases and cardiovascular diseases.

The wax myrtle leaves and twigs are pulverized into a powder and are fractionated by a number of standard techniques. The inhibition of endothelin receptor, oxidative stress and activation of NF-kB by the plant extracts after each purification step is measured and the fractions with the enriched inhibitory activity are combined. The wax myrtle leaves and twigs are extracted with an organic solvent such as methanol, ethanol, ethyl acetate, and chloroform and the extract is concentrated and washed with a water insoluble solvent such as sodium carbonate, sodium bicarbonate, and potassium hydroxide. The isolated extract is further fractionated by a myriad of standard techniques such as by solvent-solvent extraction, silica gel column chromatography, reverse phase liquid chromatography etc. to yield several fractions with different profiles of potent inhibitory activity against activation of NF-kB, oxidative stress (activation of Nerf2) and the activity of the endothelin receptor.

The assay-guided enrichment leads to plant extracts that are enriched in Myriceric acid A, B, C and D, myricadiol, taraxerol, taraxerone, and myricitrin (a flavoniod glycoside); compounds that have demonstrated inhibition of activation of NF-κB as disclosed in U.S. Application Ser. No. 60/779,142, hereby incorporated by reference.

The methods also lead to the enrichment of many triterpeniods, anthraquinone, and flavonoids that inhibit oxidative stress by the activation of the transcription factor called Nerf 2. Specifically, the plant fractions include carnosol, rosmanol, epirosmanol, isorosmanol, galdosol, rosmarinic acid, carnosic acid, miltirone, atuntzensin A, luteolin, 7-O-methyl luteolin, eupafolin, 12-O-methyl carnosol, hydroxycinnamic acid, caffeic acid, isoscutellarein 7-O-glucoside and genkwanin.

The fractions are also enriched in additional compounds that are known to inhibit endothelin receptor. These compounds include, but not limited to, Myriceric acid A, B, C and D, myricadiol, taraxerol, taraxerone, and myricitrin (a flavoniod glycoside).

The plant extracts described above may be formulated for administration in a pharmaceutical carrier in accordance with known techniques. See, e.g., Remington, The Science And Practice of Pharmacy (9^{th} Ed. 1995). In the manufacture of a pharmaceutical formulation according to the invention, the extract (including the physiologically acceptable salts thereof) is typically admixed with inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a tablet, which may contain from 0.01 or 0.5 percent to 95 percent or 99 percent by weight of the extract.

The formulations of the invention include those suitable for oral, rectal, topical, buccal (e.g., sub-lingual), vaginal, parenteral (e.g., subcutaneous, intramuscular, intradermal, or intravenous), topical (i.e., both skin and mucosal surfaces, including airway surfaces) and transdermal administration, although the most suitable route in any given case will depend on the nature and severity of the condition being treated.

Formulations suitable for oral administration may be presented in discrete units, such as capsules, cachets, lozenges, or tables, each containing a predetermined amount of the extract; as a powder or granules; as a solution or a suspension in an aqueous or non-aqueous liquid; or as an oil-in-water or water-in-oil emulsion. Such formulations may be prepared by any suitable method of pharmacy which includes the step of bringing into association of the isolated extract and a suitable carrier (which may contain one or more accessory ingredients as noted above). In general, the formulations of the invention are prepared by uniformly and intimately admixing the isolated extract with a liquid or finely divided solid carrier, or both, and then, if necessary, shaping the resulting mixture. For example, a tablet may be prepared by compressing or molding a powder or granules containing the isolated extract, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Formulations suitable for buccal (sub-lingual) administration include lozenges comprising the isolated extract in a flavored base, usually sucrose and acacia or tragacanth; and pastilles comprising the compound in an inert base such as gelatin and glycerin or sucrose and acacia.

Formulations of the present invention suitable for parenteral administration comprise sterile aqueous and non-aqueous injection solutions of the isolated extract, which preparations are preferably isotonic with the blood of the intended recipient. These preparations may contain anti-oxidants, buffers, bacteriostats and solutes which render the formulation isotonic with the blood of the intended recipient. Aqueous and non-aqueous sterile suspensions may include suspending agents and thickening agents. The formulations may be presented in unitdose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described.

Formulations suitable for rectal administration are preferably presented as unit dose suppositories. These may be prepared by mixing the isolated extract with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers which may be used include petroleum jelly, lanoline, polyethylene glycols, alcohols, transdermal enhancers, and combinations of two or more thereof.

Formulations suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)).

The therapeutically effective dosage of any isolated extract, the use of which is in the scope of present invention, will vary somewhat from compound to compound, and patient to patient, and will depend upon factors such as the age and condition of the patient and the route of delivery. Such dosages can be determined in accordance with routine pharmacological procedures known to those skilled in the art. The dosages will change for the particular condition being treated. For example, a dosage of from about 5 to 40 mg/kg may be suitable for treatment of coronary heart disease, but not for use as an antifungal agent.

As a general proposition, a preferred dosage from about 20 to 35 mg/kg is believed to have therapeutic efficacy, with all weights being calculated based upon the weight of the isolated extract, including the cases where a salt is employed. Toxicity concerns at the higher level may restrict intravenous dosages to a lower level such as up to about 20 mg/kg, with all weights being calculated based upon the weight of the active base, including the cases where a salt is employed. A preferred dosage from about 30 mg/kg to about 50 mg/kg may be employed for oral administration. Typically, a preferred dosage from about 20 mg/kg to 30 mg/kg may be employed for intramuscular injection. The duration of the treatment is usually once per day for a period of two to three weeks or until the condition is essentially controlled. The isolated extract of wax myrtle can be used to treat coronary heart disease or as an antifungal and/or antibacterial agent, as an anticoagulant, cholesterol retarding agent, acne, eczema, and myocardium from hypoxia-induced contractile failure.

The following examples are provided in order to further illustrate various embodiments of the invention and are not to be construed as limiting the scope thereof.

### EXAMPLES

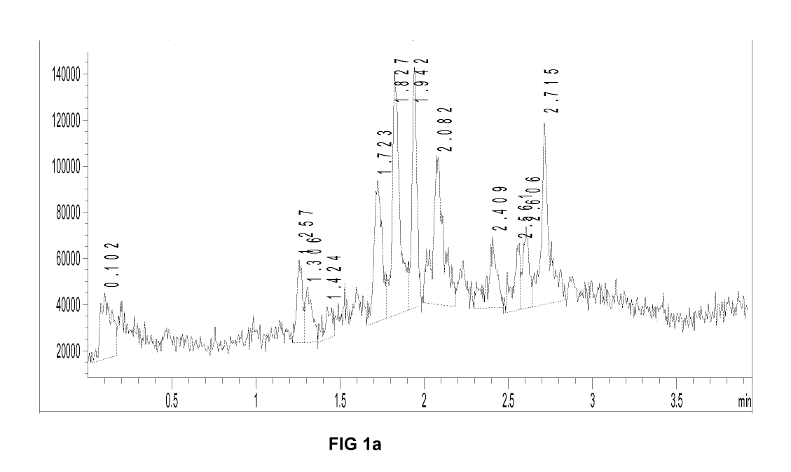

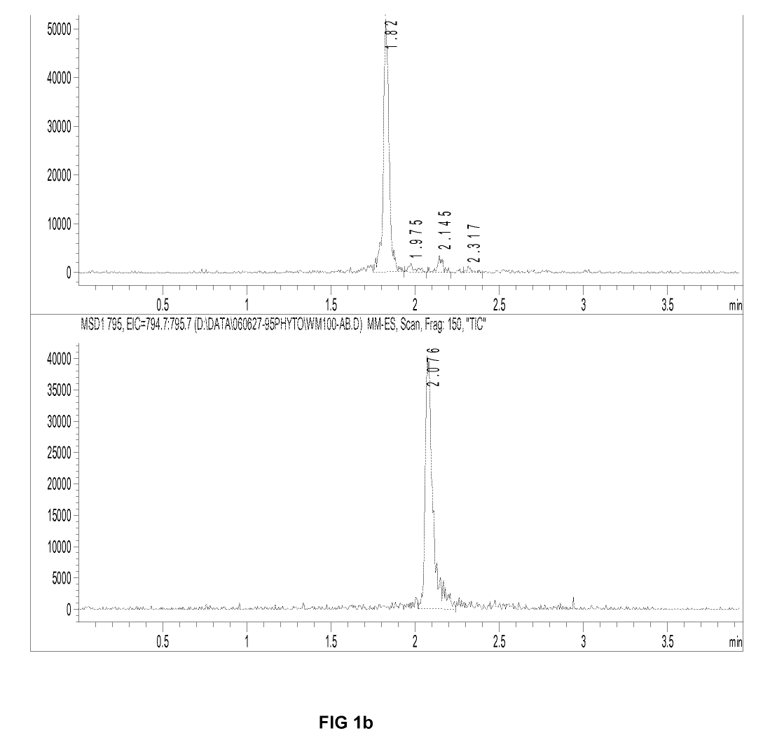

Enrichment of inhibitors of NF-kB, oxidative stress (activation of Nerf2) and endothelin receptor A from wax myrtle: Dried leaves and twigs of wax myrtle were extracted with methanol and enriched on a column chromatography to obtain a fraction highly enriched in myriceric acid A and myriceric acid C (FIGS. 1a and 1b Finger Print of ENRICHED EXTRACT, LC-MS HPLC Profile: Intensity (mV, y-axis) retention times (x-axis). Briefly, the Wax myrtle leaves and twigs were cut into small pieces and were dried in a drying oven at 45 C to remove all traces of moisture. The dried materials were stored at −20 C freezers before processing. The dried samples were Pulverized using Wiley mill with appropriate filters to obtain 20-40 micron particle size. Again, the samples were stored at −20 C in air tight zip-lock bags. About 50 gram of sample was systematically extracted on a Soxhlet extractor using methanol. The methanol fraction was dried using flash roto-evaporator to remove all solvents and dried samples were frozen and freeze-dried to obtain completely dry powder. The powder was transferred to pre-weighed scintillation vials and stored at −20 C. The methanol crude extract was subjected to flash column chromatography using Chloroform-methanol gradient and the pooled fraction (Fractions 15-17) rich with Myriceric acid A and Myriceric acid C was designated as enriched extract and subjected to fingerprinting on an LC-MS.

Identification of active ingredients: The enriched extract was re-chromatographed on a hypersil C18 reversed phase column (100×2.1 mm, 5 μm), eluted with water-acetonitrile gradient with a flow rate of 0.6 ml/min. The sample was monitored based on the UV character as well as by mass detection. For LC, LC pumps: Shimadzu LC-6 binary high-pressure system with CTC PAL injector was used. C18 (reversed phase) Agilent Zorbax XDB column with a dimension, 21.2×100 mm. The solvent system consisted of water with 0.05% TFA and Methanol with 0.05% TFA. The injection volume: 500 uL. The fractions were collected using a fraction collector (Advantec set to time based fractionation starting at 0.8 min with 0.20 min collection steps). The LC/MS/MS data was acquired for ENRICHED EXTRACT using both electrospray positive and negative ion detection. The LC/MS detection included UV and total ion current chromatogram for the electrospray positive ion LC/MS/MS analysis. LC/MS mass spectra for the most intense peaks in the chromatogram were analyzed. The LC/MS/MS data acquired from the standard of Myriceric acid A and C using the same conditions were used to quantify Myriceric acid A and C in the standardized enriched extract. The Wax myrtle leaves are highly enriched in myriceric acid A. Very high concentrations of myriceric acid C were also detected in enriched extract (FIG. 1). Various other related compounds, including myriceric acid B, myricerol, and ursomyr were also detected.

Biological Evaluation of Wax Myrtle Enriched Extract:

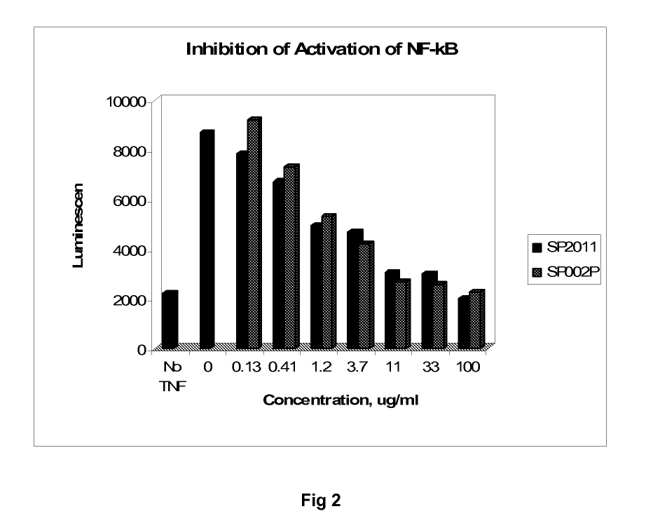

A dose-dependent decrease in transactivation of NF-kB by ENRICHED EXTRACT was also observed (FIG. 2). It is noteworthy that the inhibition of the activation of NF-kB by myriceric acid and enriched extract was very similar (FIG. 2 Inhibition of TNF-□ mediated induction of luciferase activity in A549-NF-□B stable reporter cell line by SP 2011 and SP002P). This could be due to the positive interactions between myriceric acid and other components present in the Wax Myrtle extract.

Myriceric Acid A is a potent inhibitor of ET-1-induced [Ca⁺⁺] flux in rat aortic smooth muscle cells A7r5 (IC₅₀=7.5 ng/ml or 11 nM) and the binding of radiolabeled ET to rat aortic smooth muscle cells (Kᵢ=66 nM or 40 ng/ml) (23) enriched extract has about 20% of Myriceric Acid A (FIG. 1) and as expected, it inhibited binding of ¹²⁵I-ET-1 to ET_{A}. Inhibition of specific binding of ¹²⁵I-ET-1 to ET_{A} was determined by using CHO membranes as described earlier. BQ-123, commercially available ET_{A} antagonist was used as an internal control. CHO cell membrane preparations over expressing ET_{A} were obtained from Amersham (Piscataway, N.J., USA and ¹²⁵I-ET-1 (15,000 cpm) was used to perform ET-receptor radioligand binding assay as described previously. Three different concentrations (100 □g/ml, 10 □g/ml, and 1 □g/ml) of the methanol extract and enriched extract were tested in triplicates. A complete inhibition of specific binding of ¹²⁵I-ET-1 to ET_{A} by enriched extract at all three different concentrations was observed. We are currently evaluating the inhibition of binding of ¹²⁵I-ET-1 to ET_{A} by enriched extract at concentrations below one μg/ml.

Inhibition of Proliferation of Aortic Smooth Muscle Cells (A7r5) by wax myrtle extract: The Methylene Blue Cell Proliferation assay assess growth inhibition in a cell-based proliferation assay using protein staining as an estimate of relative cell number. For assessment of cell-based potency, A7r5 (20,000 cells/well) were plated in 96-well plates in the growth media. After 24 h, cells were exposed to compounds at different concentrations. A final concentration of 0.2% DMSO was added to the growth media to keep the compounds in solution. After 3 days, relative cell numbers were estimated using methylene blue staining. The absorbance at 620 nm was read in a microplate reader. Data were analyzed using curve-fitting macros written for Microsoft Excel. Concentrations with IC₅₀ were interpolated using the equation: y=Vₘₐₓ×[1−(xⁿ/(Kⁿ+xⁿ))], where “K” is equal to IC₅₀.

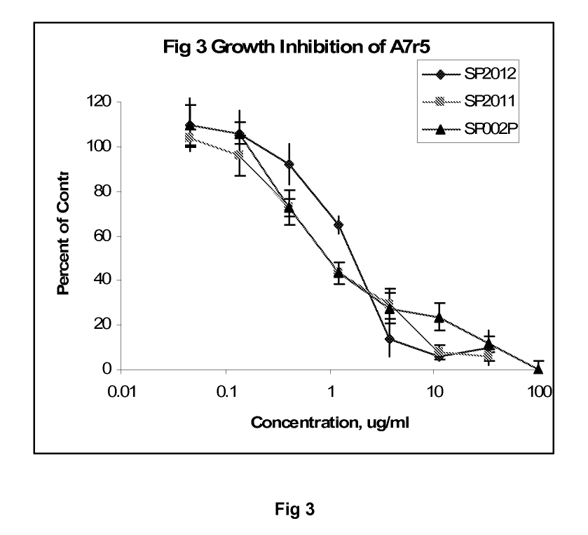

Results in FIG. 3 (Growth Inhibition of Aortic Smooth Muscle Cells (A7r5) by ENRICHED EXTRACTWM extract, Myr. A and Myr. C Grown in Serum Free Medium as Determined) show that wax myrtle extract, myriceric acid A and Myriceric Acid C resulted in a dose-dependent inhibition of cell growth in A7r5 . The estimated IC₅₀ for wax myrtle extract=0.72±0.2 □μg/ml, Myriceric acid A=0.87±0.30 □μg/ml and for Myriceric acid C=2.3±0.7 □g/ml. Interestingly, the inhibition of the growth of A7r5 by myriceric acid and wax myrtle extract were not significantly different.

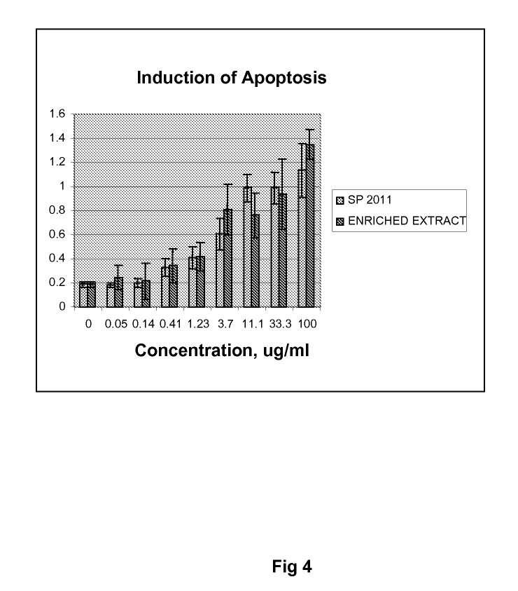

Induction of Apoptosis in Aortic Smooth Muscle Cells (A7r5) by wax myrtle extract and myriceric acid,: Quantitation of apoptotic cell death was determined by Cell Death ELISA (Roche Biochemicals) that measures cytoplasmic histone-DNAfragments produced during apoptosis. Briefly, A7r5 cells (5×10³/well)were grown in 96-well plates and treated, in triplicates, for 24 h with the indicated doses of wax myrtle extract and myriceric acid A. After treatment, the 96-well plates were centrifuged (200×g) for 10 min. The supernatant was discarded, lysis buffer was added, and samples were incubated at room temperature. Lysates were transferred to reaction wells and incubated with anti-histone biotin and anti-DNA peroxidase antibodies and incubated at room temperature for 2 h. After three washes, the peroxidase substrate was added to each well, and the plates were read at 405 nm after 15-min incubation. The enrichment of histone-DNAfragments treated cells is expressed as absorbance at 405 nm. As evident by the enrichment of DNA fragments, the treatment of A7r5 cells with ENRICHED EXTRACT and myriceric acid resulted in a similar dose-dependent apoptosis (FIG. 4 Induction of Histone-DNA Fragments in Aortic Smooth Muscle Cells (A7r5) by ENRICHED EXTRACT, SP 2011, Grown in Serum Free Medium).

In summary, the ‘Preliminary Results’ demonstrate that ENRICHED EXTRACT is highly enriched in myriceric acid A, myriceric acid C and other potent inhibitors of ET-signaling pathway. Data suggest that myriceric acid A belongs to a new class of ET antagonists that not only inhibit ET_{A} but also inhibit the expression of ET-1 and ET_{A}, thus resulting in more effective block of ET signaling pathway (I₅₀=11 nM). These effects are mediated through the dual inhibition of ET_{A} and inhibition of activation of NF-□B. ENRICHED EXTRACT induces apoptosis, and inhibits cell proliferation of rat aortic smooth muscle cells suggesting that it may be able to induce apoptosis in the pulmonary artery walls and reduce obstructive vascular remodeling in PAH patients. ENRICHED EXTRACT further warrants evaluation in vivo for the treatment of PAH.

That which is claimed:

1. A method for the treatment or prevention of cancer, inflammation, cardiovascular diseases, coronary heart disease comprising administering to a person in need of such treatment or prevention a therapeutically effective amount of an enriched fraction from wax myrtle.

2. A method for preparing an isolated extract from wax myrtle that is enriched in an inhibitor of activation of NF-kB.

3. A method according to claim 2, wherein the inhibitor of activation of NF-kB is enriched from the group consisting of myrirceric acid A, B, C and D. and, myricadiol, taraxerol, taraxerone, and myricitrin (a flavoniod glycoside).

4. A method for preparing an isolated extract from wax myrtle that is enriched in an inhibitor of oxidative stress.

5. The method of claim 4, wherein the inhibitor of activation of oxidative stress is enriched from the group consisting of carnosol, rosmanol, epirosmanol, isorosmanol, galdosol, rosmarinic acid, carnosic acid, miltirone, atuntzensin A, luteolin, 7-O-methyl luteolin, eupafolin, 12-O-methyl carnosol, hydroxycinnamic acid, caffeic acid, isoscutellarein 7-O-glucoside and genkwanin

6. A method for preparing an isolated extract from wax myrtle that is enriched in an inhibitor of endothelin receptor.

7. The method of claim 6, wherein the inhibitor of endothelin receptor is enriched from the group consisting of myriceric acid A (myriceron caffeoyl ester), myriceric acid B, myriceric acid C, and myriceric acid D.
